(19) United States
(12) Patent Application Publication
(10) Pub. No.: US 2023/0005587 A1
(43) Pub. Date: Jan. 5, 2023

(54) DETERMINING WHETHER ADJUSTMENTS OF INSULIN THERAPY RECOMMENDATIONS ARE BEING TAKEN INTO ACCOUNT

(71) Applicant: Bigfoot Biomedical, Inc., Milpitas, CA (US)

(72) Inventors: Alexandra Elena Constantin, San Jose, CA (US); Zahra Eghtesadi, Milpitas, CA (US)

(21) Appl. No.: 17/756,586

(22) PCT Filed: Nov. 30, 2020
(86) PCT No.: PCT/US2020/070829

§ 371 Date: May 27, 2022

Related U.S. Application Data
(60) Provisional application No. 62/943,147, filed on Dec. 3, 2019.

(51) Int. Cl. G16H 20/17

(52) U.S. Cl. CPC G16H 20/17

(57) ABSTRACT

A method comprises: receiving, using a computer system and for a person with diabetes for whom amounts of insulin ingestion is not detected, blood glucose values for a time period, wherein a first changed insulin dosage recommendation was issued to the person with diabetes during the time period; determining, using the computer system and based on the blood glucose values, whether the person with diabetes has taken into account the first changed insulin dosage recommendation in administering insulin; and taking an action based on the determination.

### CROSS-REFERENCE TO RELATED APPLICATIONS

This application claims priority to U.S. Provisional Application No. 62/943,147, filed on Dec. 3, 2019, entitled “DETERMINING WHETHER ADJUSTMENTS OF INSULIN THERAPY RECOMMENDATIONS ARE BEING TAKEN INTO ACCOUNT”, the disclosure of which is incorporated by reference in its entirety.

### TECHNICAL FIELD

This document relates to determining whether adjustments to insulin therapy recommendations are being taken into account.

### BACKGROUND

Diabetes mellitus is a chronic metabolic disorder caused by the inability of a person's pancreas to produce sufficient amounts of the hormone insulin such that the person's metabolism is unable to provide for the proper absorption of sugar and starch. This failure leads to hyperglycemia, i.e., the presence of an excessive amount of glucose within the blood plasma. Persistent hyperglycemia has been associated with a variety of serious symptoms and life threatening long-term complications such as dehydration, ketoacidosis, diabetic coma, cardiovascular diseases, chronic renal failure, retinal damage and nerve damages with the risk of amputation of extremities. Because healing is not yet possible, a permanent therapy is necessary which provides constant glycemic control in order to constantly maintain the level of blood analyte within normal limits. Such glycemic control is achieved by regularly supplying external drugs to the body of the patient to thereby reduce the elevated levels of blood analyte. An external biologically effective drug (e.g., insulin or its analog) is commonly administered by means of daily injections. In some cases, multiple, daily injections of a mixture of rapid- and long-acting insulin are administered via a reusable transdermal liquid dosing device.

### SUMMARY

In a first aspect, a method comprises: receiving, using a computer system and for a person with diabetes for whom amounts of insulin ingestion is not detected, blood glucose values for a time period, wherein a first changed insulin dosage recommendation was issued to the person with diabetes during the time period; determining, using the computer system and based on the blood glucose values, whether the person with diabetes has taken into account the first changed insulin dosage recommendation in administering insulin; and taking an action based on the determination.

Implementations can include any or all of the following features. The determination indicates that the person with diabetes has taken into account the first changed insulin dosage recommendation, and wherein the action comprises generating a second changed insulin dosage recommendation to the person with diabetes. The determination indicates that the person with diabetes has not taken into account the first changed insulin dosage recommendation, and wherein the action comprises preventing a second changed insulin dosage recommendation from being generated to the person with diabetes. The determination indicates that the person with diabetes has not taken into account the first changed insulin dosage recommendation, and wherein the action comprises generating a communication without a second changed insulin dosage recommendation. The communication is generated to the person with diabetes or to a physician. The blood glucose values include a first blood glucose profile from before the first changed insulin dosage recommendation was generated, and a second blood glucose profile from after the first changed insulin dosage recommendation was generated. The method further comprises determining, based on the first blood glucose profile and the second blood glucose profile, percentiles of the blood glucose values for the time period. Determining whether the person with diabetes has taken into account the first changed insulin dosage recommendation in administering insulin comprises applying an equality test to the first blood glucose profile and the second blood glucose profile. Determining whether the person with diabetes has taken into account the first changed insulin dosage recommendation in administering insulin comprises using a trained classifier. Determining whether the person with diabetes has taken into account the first changed insulin dosage recommendation in administering insulin comprises applying a hypothesis test to the blood glucose values. Applying the hypothesis test comprises fitting parameters of a log-normal distribution to the blood glucose values. Determining whether the person with diabetes has taken into account the first changed insulin dosage recommendation in administering insulin comprises looking for a change in the blood glucose values opposite to a direction of the first changed insulin dosage recommendation. The method further comprises: predicting a first total daily basal dose (TDBD) mismatch value using at least some of the blood glucose values, the first TDBD mismatch value corresponding to time before the first changed insulin dosage recommendation; predicting a second TDBD mismatch value using at least some of the blood glucose values, the second TDBD mismatch value corresponding to time after the first changed insulin dosage recommendation; and determining whether the first and second TDBD mismatch values are different from each other, wherein the determination whether the person with diabetes has taken into account the first changed insulin dosage recommendation in administering insulin is based on the determination whether the first and second TDBD mismatch values are different from each other. The method further comprises performing a hypothesis test to determine whether the first and second TDBD mismatch values are different from each other.

In a second aspect, a method comprises: generating, using a computer system, a first set of blood glucose profiles corresponding to first persons with diabetes who do not take into account a changed insulin dosage recommendation in administering insulin; generating a second set of blood glucose profiles corresponding to second persons with diabetes who take into account the changed insulin dosage recommendation in administering insulin; and training a classifier using the computer system, the classifier trained based on the first set of blood glucose profiles and the second set of blood glucose profiles.

Implementations can include any or all of the following features. The method further comprises obtaining the first set of blood glucose profiles and the second set of blood glucose profiles by simulation. The method further comprises performing feature engineering regarding the first set of blood glucose profiles and the second set of blood glucose profiles, the feature engineering taken into account in training the classifier. The method further comprises applying an equality test to the first set of blood glucose profiles and the second set of blood glucose profiles, and designating an outcome of the equality test as a feature in training the classifier.

In a third aspect, a method comprises: generating, using a computer system, a first set of blood glucose profiles corresponding to first persons with diabetes who do not take into account a changed insulin dosage recommendation in administering insulin; generating a second set of blood glucose profiles corresponding to second persons with diabetes who take into account the changed insulin dosage recommendation in administering insulin; and applying, using the computer system, a hypothesis test to the first set of blood glucose profiles and the second set of blood glucose profiles.

Implementations can include any or all of the following features. Applying the hypothesis test comprises fitting parameters of a log-normal distribution to the first set of blood glucose profiles and the second set of blood glucose profiles. The method further comprises training a classifier based on an outcome of applying the hypothesis test. The method further comprises obtaining the first set of blood glucose profiles and the second set of blood glucose profiles by simulation.

### BRIEF DESCRIPTION OF DRAWINGS

FIG. 1 shows an example of a system where compliance with insulin therapy recommendations can be evaluated.

FIGS. 2A-B show examples of graphical user interfaces.

FIG. 3 shows a simplified block diagram of a computing platform for evaluating insulin recommendation compliance.

FIG. 4 shows an example of the compliance evaluation system in FIG. 1.

FIG. 5 shows a simplified block diagram of an example of a system for insulin-based management of diabetes.

FIG. 6 shows a functional block diagram of a system for training a compliance classifier using machine learning, in accordance with one or more embodiments.

FIG. 7 shows an example of a method.

FIG. 8 shows an example of a computer device that can be used to implement the techniques described here.

Like reference symbols in the various drawings indicate like elements.

### DETAILED DESCRIPTION

This document describes examples of systems and techniques for determining whether adjustments to insulin therapy recommendations are being taken into account. In some implementations, a computer-based approach can be applied for determining whether a person with diabetes is at least considering a received communication regarding a change in an insulin therapy setting. For example, a trained classifier, equality testing, and/or hypothesis testing can be applied to determine whether the adjustment recommendations are being taken into account. In some implementations, determining whether the person with diabetes has taken into account a changed insulin dosage recommendation in administering insulin can include looking for a change in blood glucose values that is opposite to a direction of the first changed insulin dosage recommendation. For example, if the changed insulin dosage recommendation includes an increase of insulin, the determination can evaluate whether the blood sugar levels have decreased. As another example, if the changed insulin dosage recommendation includes a decrease of insulin, the determination can evaluate whether the blood sugar levels have increased.

Insulin delivery devices include, but are not limited to, insulin injection pens, insulin inhalers, insulin pumps, and insulin syringes. The improper dosing of insulin, whether due to human error, malfunction of an insulin pen, skipping doses, double dosing, or incorrect dosing, is always a concern. Methods, devices, and systems provided herein are described for the delivery of insulin, collection of blood glucose data, and/or the treatment of diabetes. Moreover, methods, devices, and systems provided herein may be adapted for the delivery of other medications, the collection of other analyte data, and/or the treatment of other diseases. Methods, devices, and systems provided herein are described by exemplifying features and functionalities of a number of illustrative embodiments. Other implementations are also possible.

Some examples herein refer to an insulin injection pen. An insulin injection pen includes at least one container holding insulin (e.g., an insulin cartridge), a dial or other mechanism to specify a dose, and a pen needle for transcutaneous delivery of the insulin into the tissue or vasculature of the person with diabetes. In a reusable insulin injection pen, the insulin container (e.g., the cartridge) is replaceable or refillable. A prefilled insulin injection pen is intended for use during a limited time. The dose-specifying mechanism can include a rotatable wheel coupled to mechanics and/or electronics for capping the administered amount of insulin at the volume of the specified dose (e.g., in terms of a number of units of insulin). The dose-specifying mechanism can have a mechanical and/or electronic display that reflects the current setting of the mechanism. The pen needle can be permanently attached to the housing of the insulin injection pen (e.g., as in the case with a disposable pen), or it can be removable (e.g., so that a new needle can be applied when needed). For example, a replaceable pen needle can include a hollow needle affixed to a fitting configured for removeable attachment to an end of the insulin injection pen.

Some examples herein refer to a mobile communication device. As used herein a mobile communication device includes, but is not limited to, a mobile phone, a smartphone, a wearable electronic device (e.g., a smart watch), a tablet, a laptop computer, a portable computer, and similar devices. A mobile communication device includes one or more processors, a non-transitory storage device (e.g., a memory and/or a hard drive) holding executable instructions for operating the mobile communication device, wireless communication components, and one or more input and/or output devices (e.g., a touchscreen, display, or keyboard). The mobile communication device can operate according to one or more application programs stored locally on the mobile communication device, or remotely (e.g., when using cloud computing), or combinations thereof. The mobile communication device can execute at least one operating system in order to perform functions and services.

Some examples herein refer to a continuous glucose monitor (CGM). A CGM is an electronic device configured to take readings of glucose values on an ongoing basis or at regular intervals in order to estimate the blood glucose level of the person with diabetes. Some CGMs determine blood glucose values periodically (e.g., after a certain number of seconds or minutes) and output the information automatically or upon being prompted. The CGM may include wireless communication components for one or more types of signaling, including, but not limited to, Near-Field Communication (NFC) and/or Bluetooth communication.

In some embodiments, systems, devices, and methods provided herein can recommend insulin doses (e.g., dosages of long-acting and/or rapid-acting insulin) using any suitable technique. In some embodiments, recommended insulin dosages may be based upon blood glucose data (e.g., current estimated glucose value (EGV) from a CGM, flash glucose monitor, blood glucose meter, or any other sensor, blood glucose trend data, etc.), insulin administration data (bolus dosage amounts of rapid-acting insulin, dosages of long-acting insulin, dosage times, calculation of Insulin-on-Board (“IOB”) and/or active insulin, etc.), meal data (mealtimes, user estimated carbohydrates, user estimated meal categorizations, user estimated glycemic impact of meal user meal history, user meal trends, etc.), and/or one or more insulin delivery parameters (e.g., total daily dose of basal insulin or long-acting insulin, carbohydrate-to-insulin ratio (CR), insulin sensitivity factor (ISF), etc.). Methods, devices and systems provided herein can, in some embodiments, adjust insulin delivery parameters over time based on glucose data and/or insulin administration data.

Some examples herein refer to long-acting insulin and rapid-acting insulin, or in some cases more generally to first and second types of insulin. Insulin used for therapeutic treatment is often synthesized human insulin. Moreover, different insulins can be characterized in how quickly they typically begin to work in the body of the person with diabetes after administration, and/or how long they typically remain active in the body of the person with diabetes. Rapid-acting insulin can be used to dose for meals or to correct high blood sugar. There is more than one type of insulin that can be considered a rapid-acting insulin. Many, but not necessary all, rapid-acting insulins begin working within about one hour after administration. Similarly, there is more than one type of insulin that can be considered a long-acting insulin, and many, but not necessarily all, long-acting insulins begin working about one hour or longer after administration. Long-acting insulin is often referred to as basal insulin (e.g., insulin used to support basic metabolic needs). Generally, a long-acting insulin has a greater active time (i.e., the length of time that the insulin continues to be active in the body of the person with diabetes after administration) than a rapid-acting insulin. As such, a long-acting insulin is an example of a type of insulin having an active time that is longer than an active time for a type of insulin such as a rapid-acting insulin.

Examples herein refer to TDBD. The TDBD can reflect a setting of an insulin therapy corresponding to the amount of long-acting insulin to be administered. The actual TDBD of a person with diabetes may not be known. Rather, a programmed TDBD value can be entered into a diabetes therapy management system for the person and can be adjusted as needed. A TDBD mismatch value, moreover, can represent a determination of a relationship between the programmed TDBD value and the unknown actual TDBD value. In some implementations, the determined TDBD mismatch value represents a division of the programmed TDBD value with the unknown actual TDBD value. For example, a new (i.e., adjusted) TDBD value can be calculated by dividing the programmed TDBD value by the determined TDBD mismatch value.

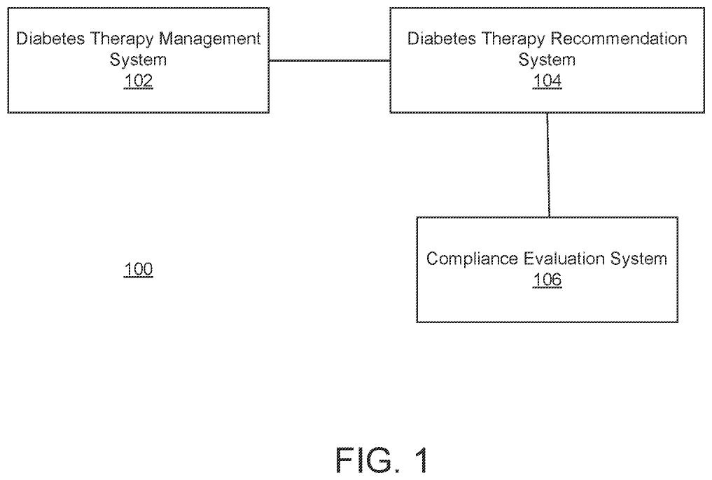

FIG. 1 shows an example of a system 100 where compliance with insulin therapy recommendations can be evaluated. The system 100 can be used with one or more other examples described elsewhere herein. The system 100 includes a diabetes therapy management system 102. In some implementations, the diabetes therapy management system 102 is configured for use by a person with diabetes for monitoring, controlling, and/or documenting the treatment of diabetes. The diabetes therapy management system 102 can include one or more components. For example, the diabetes therapy management system 102 can be or include an insulin injection pen, a pen cap for an insulin injection pen, a CGM, and/or a smartphone having software installed. That is, the diabetes therapy management system 102 may not automatically administer doses of insulin to the person; rather, the diabetes therapy management system 102 may inform the person about available data and/or recommendation to aid the person in deciding whether to ingest insulin and in what amount. Moreover, the diabetes therapy management system 102 may not directly measure the amount of insulin ingested by the person.

The system 100 includes a diabetes therapy recommendation system 104. In some implementations, the diabetes therapy recommendation system 104 is configured to exchange information with the diabetes therapy management system 102, such as by wired and/or wireless communication. For example, the diabetes therapy recommendation system 104 can receive blood glucose values regarding the person with diabetes from the diabetes therapy management system 102 at periodic intervals or at random times, and can generate one or more diabetes-related recommendations based on the blood glucose values to the diabetes therapy management system 102. If the diabetes therapy management system 102 does not directly measure the amount of insulin ingested by the person, the diabetes therapy recommendation system 104 does not have direct information about what dosage the person is actually ingesting.

The system 100 includes a compliance evaluation system 106. In some implementations, the compliance evaluation system 106 is configured to exchange information with the diabetes therapy recommendation system 104, such as by wired and/or wireless communication. For example, compliance evaluation system 106 can receive processable data regarding the person with diabetes (e.g., blood glucose values) from the diabetes therapy recommendation system 104, and can provide one or more determinations of recommendation compliance based on the processable data.

One or more user interfaces can be presented to a person with diabetes. In some implementations, the diabetes therapy management system 102 can provide a user interface for presenting one or more recommendations. For example, the recommendation can be presented on a display or screen, automatically or upon request.

A user interface can depict insulin data. In some cases, the user interface can indicate an amount insulin on board (JOB) in an insulin ingestion device (e.g., an insulin injection pen or a pump), which may be only for a particular type of insulin (including, but not limited to, rapid-acting insulin). In some implementations, IOB (sometimes referred to as active insulin) can be defined as the amount of insulin that has been delivered and is still active in the person's body based on estimated duration of insulin action. In some cases, an IOB calculation may be for both rapid-acting and long-acting insulin. In some cases, the user interface can display information, including, but not limited to, the time and/or amounts of the most recent doses of quick-acting and/or long-acting insulins. In some cases, the user interface can permit the user to click on the insulin data or otherwise navigate in the mobile app to more detailed and/or more complete insulin delivery data. In some cases, a user interface can overlay blood glucose data and insulin delivery data in any suitable format, such as a graphical display of the timing of blood glucose data vs. the timing of insulin delivery data.

In use, a user (e.g., the person with diabetes and/or a caregiver) can use the diabetes therapy management system 102 to get recommendations regarding an appropriate insulin dosage. In the case of an upcoming need to deliver long-acting insulin, a text can be presented that provides a recommended long-acting insulin dosage. In some cases, a recommended dosage may appear on a pen cap of an insulin injection device. In the case of the user wanting to deliver a bolus of rapid-acting insulin, the user may press a bolus calculator button on the diabetes therapy management system 102 to enter into a bolus calculator. Any suitable bolus calculator could be used in systems, methods, and devices provided herein. For example, the bolus calculator can provide a user interface for a user to enter a meal announcement as either a correction only, a small meal, a normal sized meal, or a large meal. Upon selecting the meal size, the user interface can provide a recommended bolus dosage based on a number of carbohydrates associated with the corresponding button and optionally based upon blood glucose data. Additionally, or alternatively, dose capture pen caps for the insulin injection pen can include a user interface that permits the user to announce a meal size, including, but not limited to, a small meal, medium meal, or large meal.

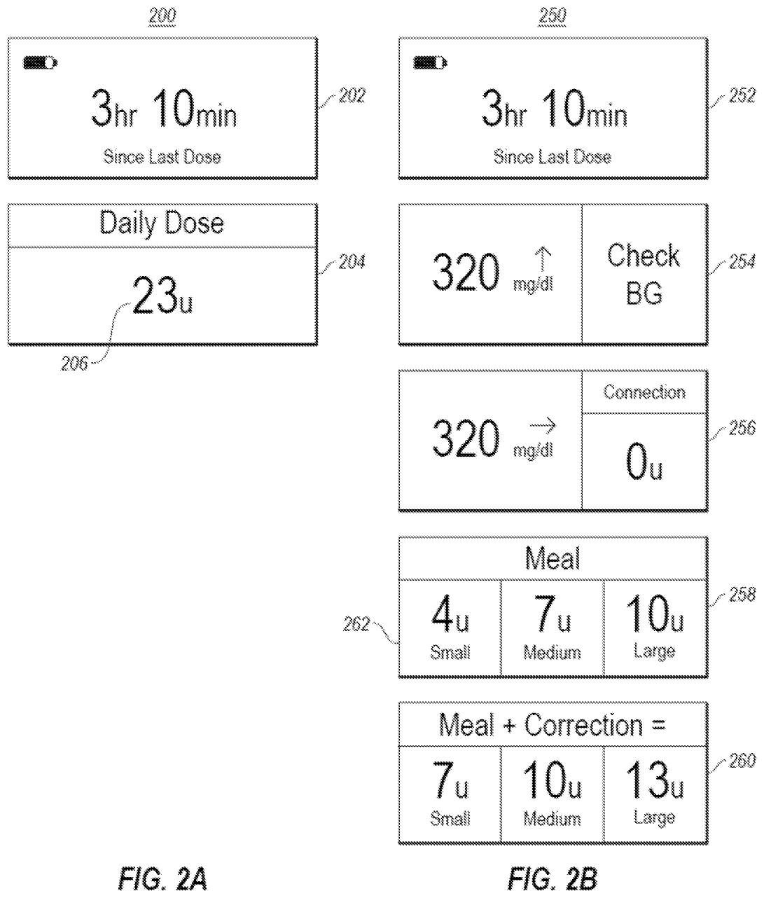

FIGS. 2A-B show examples of graphical user interfaces 200 and 250, respectively. The graphical user interfaces 200 and/or 250 can be used in combination with one or more other examples described elsewhere herein. Each of the graphical user interfaces 200 and 250 can be presented on a suitable display, including, but not limited to, a display of the diabetes therapy management system 102. In some implementations, the graphical user interface 200 relates to an insulin injection pen for a certain type of insulin, such as a long-acting insulin pen. For example, the graphical user interface 200 can be presented on a display of a pen cap for the certain type of insulin. In some implementations, the graphical user interface 250 relates to an insulin injection pen for a certain other type of insulin, such as a rapid-acting insulin pen. For example, the graphical user interface 250 can be presented on a display of a pen cap for the certain other type of insulin.

One or more of the graphical user interfaces 200 and 250 can support one or more types of processes relating to insulin therapy management. In some implementations, a pairing process can be supported that seeks to establish a connection between the pen cap and one or more other devices. For example, connection between the pen cap and a smartphone can be established. In some implementations, a daily use process can be supported that corresponds to the person's with diabetes ordinary use of the pen cap, after one or more successful pairings with the other device(s), to manage diabetes therapy.

In some implementations, a daily use workflow for the graphical user interface 200 can include a default screen (e.g., presenting a name of the insulin currently contained in the insulin injection pen), a timer screen 202, and/or a dose suggestion screen 204. For example, the person with diabetes can cycle through two or more screens using an input control (e.g., of the diabetes therapy management system 102 in FIG. 1). For example, the graphical user interface 200 can automatically return to presenting the default screen if no action is undertaken using the pen cap (e.g., by way of a button other otherwise) within a predefined time period.

In some implementations, the timer screen 202 presents a time that has elapsed since the last dose (e.g., since a most recent capping/uncapping event that qualifies as an indicator of insulin dosage). For example, the timer screen currently states that it has been 3 hours and 10 minutes since the last dose. In some implementations, the elapsed time can be based on actual dosage timing (e.g., as reported by an injection device), or on a proxy for insulin injection timing other than capping/uncapping.

In some implementations, the dose suggestion screen 204 presents an insulin dosage suggestion 206. The insulin dosage suggestion 206 can contain one or more recommendations to the person with diabetes. For example, the insulin dosage suggestion 206 currently suggests that the person with diabetes should ingest a daily dose of insulin (e.g., long-acting insulin) corresponding to an amount of 23 u (units).

In some implementations, a daily use workflow for the graphical user interface 250 can include a default screen (e.g., presenting a name of the insulin currently contained in the insulin injection pen), a timer screen 252, a blood glucose value screen 254, a blood glucose value screen 256, a dose suggestion screen 258, and/or a dose suggestion screen 260. For example, the person with diabetes can cycle through two or more screens using an input control. For example, the graphical user interface 250 can automatically return to presenting the default screen if no action is undertaken using the pen cap (e.g., by way of a button other otherwise) within a predefined time period.

In some implementations, the timer screen 252 presents a time that has elapsed since the last dose (e.g., since a most recent capping/uncapping event that qualifies as an indicator of insulin dosage). For example, the timer screen currently states that it has been 3 hours and 10 minutes since the last dose. In some implementations, the elapsed time can be based on actual dosage timing (e.g., as reported by an injection device), or on a proxy for insulin injection timing other than capping/uncapping.

In some implementations, the blood glucose value screens 254 and/or 256 presents data obtained by way of a glucose monitor or another glucose sensor (e.g., a CGM). For example, the blood glucose value screen 254 can present a value of a blood glucose estimate (e.g., 320 mg/dL), present a trend indication (e.g., an arrow pointing up, or towards the right and up, or to the right, or towards the right and down, or down), and/or a suggestion that the person with diabetes should check his or her blood glucose (BG). For example, the blood glucose value screen 256 can present a value of a blood glucose estimate (e.g., 320 mg/dL), present a trend indication (e.g., an arrow pointing up, or towards the right and up, or to the right, or towards the right and down, or down), and an insulin dosage suggestion as to whether the person with diabetes should ingest a correction dose (e.g., here zero units is recommended).

In some implementations, the dose suggestion screen 258 presents an insulin dosage suggestion 262 relating to a meal. The insulin dosage suggestion 262 can contain one or more recommendations to the person with diabetes based on information about the meal. For example, the insulin dosage suggestion 262 currently suggests that the person with diabetes should ingest 4 units of insulin if the meal is a small meal, 7 units if the meal is a medium meal, and 10 units if the meal is a large meal. In some implementations, the pen cap for which the graphical user interface 250 is implemented may work in combination with one or more glucose monitors or another glucose sensor. For example, the graphical user interface 250 can work with a CGM. As another example, the graphical user interface 250 can work with a blood glucose meter.

The graphical user interface 200 and/or 250 can present one or more other types of information not illustrated in the present examples. Such information can include, but is not limited to, messages regarding a need to charge the pen cap, messages that the pen cap is currently synchronizing data, and/or error messages.

One or more recommendations of the graphical user interface 200 and/or 250 can be updated. In some implementations, based on blood glucose data collected from the person with diabetes, the diabetes therapy recommendation system 104 (FIG. 1) can change at least one insulin dosage recommendation and provide the changed insulin dosage recommendation to the diabetes therapy management system 102.

Persons with diabetes can choose to take any of multiple approaches toward the changed insulin dosage recommendation (as well as toward the original insulin dosage recommendation.) A first category of persons may always or often follow the insulin dosage recommendation that is presented by the diabetes therapy management system 102. Such a person is likely to take the changed insulin dosage recommendation presented by the diabetes therapy management system 102 into account, and to comply with the changed insulin dosage recommendation. A second category of persons may always or often view the insulin dosage recommendation that is presented by the diabetes therapy management system 102 and use the insulin dosage recommendation in performing their own calculation of the dosage of insulin to ingest. For example, the person may usually apply a dose that is a predefined amount (e.g., in terms of unit or percentage) above or below the insulin dosage recommendation. That is, while such a person does not, or does not always, ingest exactly the recommended amount, he or she does take the changed insulin dosage recommendation presented by the diabetes therapy management system 102 into account in managing their insulin intake. A third category of persons may seldom or never follow the insulin dosage recommendation that is presented by the diabetes therapy management system 102. While such a person does ingest insulin, they are determining the dosage without the benefit of the insulin dosage recommendation available by the diabetes therapy management system 102. That is, such a person does not take the changed insulin dosage recommendation presented by the diabetes therapy management system 102 into account.

The third category of person exemplified above can merit special arrangements from the perspective of the diabetes therapy recommendation system 104 (FIG. 1). In some implementations, changes in insulin dosage recommendations may not be implemented all at once; rather, a gradual increase or decrease in the insulin dosage recommendation can occur over a period of time, such as multiple weeks or months. Moreover, the insulin dosage recommendation for the next iteration (e.g., a two-week period) is generated based on the assumption that the person has taken the amount of insulin that was recommended in the previous iteration (i.e., in the previous insulin dosage recommendation). Since the third category of person does not take the changed insulin dosage recommendation into account, their dosage of insulin may not increase/decrease according to the insulin dosage recommendation over that period of time. As a result, a discrepancy between the insulin dosage recommendations and the person's actual insulin dosage can develop over time. This may be undesirable, as a newly generated insulin dosage recommendation does not take into account the actual dosage ingested by the person with diabetes. To address this and/or other situations, one or more approaches for evaluating compliance with insulin dosage recommendations can be followed.

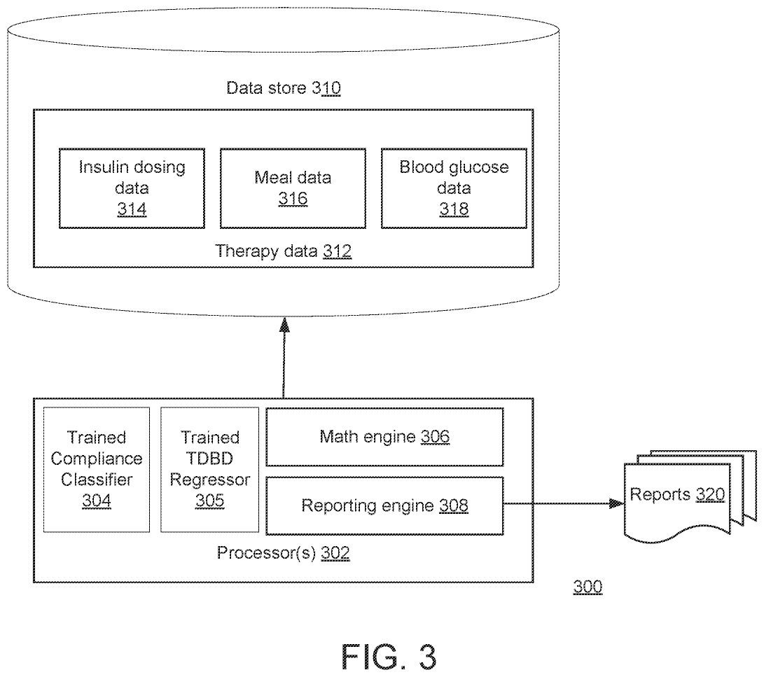

FIG. 3 shows a computing platform 300 for evaluating compliance with insulin dosage recommendations. In disclosed embodiments, computing platform 300 is, or is operative to be executed as, a data processing system, and more specifically, as a data processing system for evaluating compliance with a changed insulin dosage recommendation. Computing platform 300 may include data store 310 and processor(s) 302. Data store 310 may include therapy data 312. Therapy data 312 is data related to insulin therapy of one or more persons. In the embodiment shown in FIG. 3, therapy data 312 includes insulin dosing data 314, meal data 316, and blood glucose data 318. Alternatively, or additionally, therapy data 312 may include exercise data, sleep data, and/or physiological parameters of a patient (e.g., an insulin sensitivity factor or insulin-to-carbohydrate ratio).

Blood glucose data 318 may include data about blood glucose in a human body at one or more times. Blood glucose data 318 may include measurements of blood glucose levels, for example, raw blood glucose measurements, blood glucose estimates based on blood glucose measurements, and/or aggregations of the same (e.g., averages, trends and metrics). Blood glucose data 318 may include date and time (e.g., a timestamp), and a value for each blood glucose measurement. In disclosed embodiments, any suitable glucose sensor may provide blood glucose data 318, for example, a continuous glucose monitor (CGM), a flash glucose monitor, a blood glucose meter (BGM). In the case of CGMs and flash glucose monitors, they may be configured to provide blood glucose data 318 based on interstitial fluid glucose levels of a person, which may be correlated to blood glucose levels. A BGM may be configured to provide blood glucose data based on a blood sample. Accordingly, the term “blood glucose” is not limited herein to using just blood glucose data, values, levels etc., but is also intended to include interstitial fluid glucose levels, intermediate measurements, and legal equivalents thereof.

Insulin dosing data 314 may include dosing event data. Dosing event data may include data about insulin dosing actions at one or more times and may include, for example, a dosing time or time range, type of insulin (e.g., LA insulin and rapid acting (RA) insulin) dosed, brand of insulin, and/or amount of dosed insulin. In some embodiments, dosing event data may include an indication of a dosing mechanism, for example, injection pen, inhaler, or infusion pump. In some embodiments, dosing event data may include an indication of whether dosing event data, in part or in whole, is based on an actual dosing action (e.g., detecting insulin delivery, for example, based on a manual action of a pump or a control signal configured to cause insulin delivery), user tracking of dosing actions (e.g., a PWD or caregiver enters a dose using a therapy application executing on a mobile device), or inferred dosing actions (e.g., from capping/uncapping of an injection pen).

Processor(s) 302 may be configured to execute a number of engines for performing disclosed embodiments. In the embodiment shown in FIG. 3, processor(s) 302 includes trained compliance classifier engine 304, trained total daily basal dose (TDBD) regressor 305, math engine 306, and reporting engine 308.

Trained compliance classifier engine 304 may be configured, generally, to process therapy data 312 or part(s) of therapy data 312, and determine whether a person with diabetes has taken into account a changed insulin dosage recommendation in administering insulin. In one embodiment, a retrospective time period may be defined (e.g., as a setting), and a part of therapy data 312 processed by the trained compliance classifier engine 304 may correspond to the retrospective time period. In one embodiment, trained compliance classifier engine 304 may be a binary classifier, that is, return one of two results, a first result corresponding to “compliance detected” and a second result corresponding to “no compliance detected.” Trained compliance classifier engine 304 may assign only one label to each retrospective timestamp, for example, “compliance detected” and “no compliance detected.”

Trained TDBD regressor 305 may be configured, generally, to process therapy data 312 or part(s) of therapy data 312, and determine one or more TDBD mismatches. A part of therapy data 312 processed by the trained TDBD regressor 305 may correspond to a particular time period. In one embodiment, Trained TDBD regressor 305 may return predictions of a continuous variable, such as to determine a TDBD mismatch. Trained TDBD regressor 305 may output a determined TDBD mismatch, or can cause one or more other actions to be taken. Trained TDBD regressor 305 may be trained using one or more supervised learning techniques, including those described in more detail in this disclosure.

The TDBD can reflect a setting of an insulin therapy corresponding to the amount of long-acting insulin to be administered. The actual TDBD of a person with diabetes may not be known. Rather, a programmed TDBD value can be entered into a diabetes therapy management system for the person and can be adjusted as needed. A TDBD mismatch value, moreover, can represent a determination of a relationship between the programmed TDBD value and the unknown actual TDBD value. In some implementations, the determined TDBD mismatch value represents a division of the programmed TDBD value with the unknown actual TDBD value. For example, a new (i.e., adjusted) TDBD value can be calculated by dividing the programmed TDBD value by the determined TDBD mismatch value. The term TDBD is sometimes often applied in the context of insulin therapy administration by way of a pump device. Other terms can be used to refer to the dosage of long-acting insulin over a 24-hour period, including, but not limited to, daily long-acting insulin. As used herein, the term TDBD also includes the concept of daily long-acting insulin.

In a contemplated operation, a learning engine can train TDBD regressor 305 using training data and sets of engineered features (i.e., the feature sets in Table I) selected for model training purposes. In some embodiments, trained TDBD regressor 305 is a function or algorithm that determines a TDBD mismatch. An initial “best guess” may be used for trained TDBD regressor 305 which is then continually improved by the supervised learning engine. In disclosed embodiments, trained TDBD regressor 305 and the supervised learning engine may implement any suitable supervised learning algorithms and ensemble methods thereof for performing embodiments of the disclosure, including, for example, a linear support vector regressor (SVR), an SVR with radial basis function (RBF) kernel, Lasso, or Ridge regression model. A model training stage can be implemented with or without feature selection. In feature selection, the top n number of most relevant features can be selected for the purpose of the learning algorithm to improve model prediction and avoid over-fitting.

In disclosed embodiments, trained compliance classifier engine 304 and/or trained TDBD regressor 305 may be trained using one or more supervised and/or unsupervised learning techniques, including those described in more detail in this disclosure.

Math engine 306 may be configured to perform various statistical calculations using therapy data 312 and results provided by trained compliance classifier engine 304 and/or trained TDBD regressor 305. In various embodiments, statistical calculations may include, for example, frequency calculations, confidence calculations, probability calculations, and more.

Reporting engine 308 may be configured, generally, to generate one or more reports 320 responsive to trained compliance classifier engine 304, trained TDBD regressor 305, and/or math engine 306. Reports 320 may include descriptions of retrospective studies performed at system 100 (FIG. 1), and may include, for example, patient identifiers, descriptions of retrospective time periods, assigned class labels, the class labels and more.

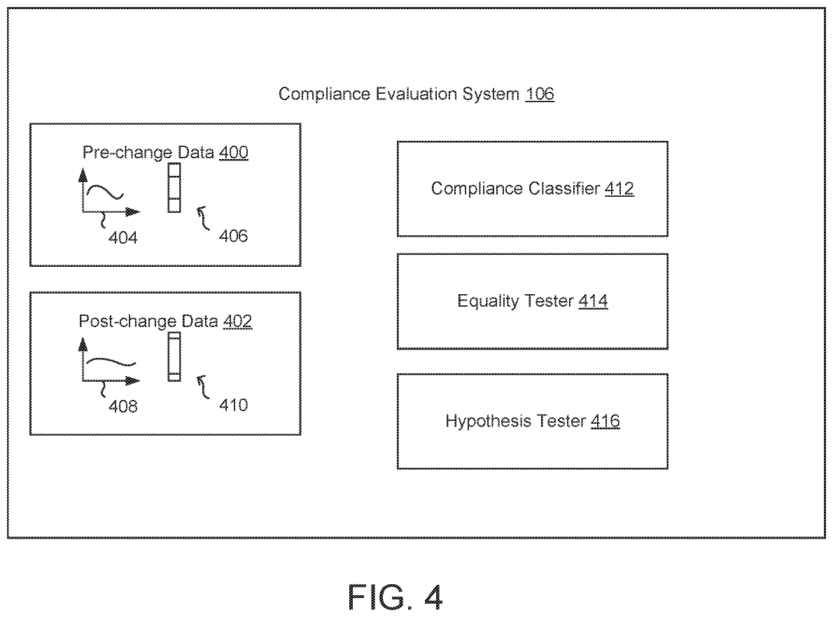

FIG. 4 shows an example of the compliance evaluation system 106 in FIG. 1. The compliance evaluation system 106 can be used with one or more examples described elsewhere herein. The compliance evaluation system 106 can collect one or more types of pre-change data 400 and one or more types of post-change data 402. The term “pre-” here refers to periods of time before a particular changed insulin dosage recommendation is generated. For example, when recommendations are updated at the beginning of a new iteration (e.g., every two weeks), the pre-change data 400 can include data from more than two weeks ago (e.g., data from 2-4 weeks back in time). The term “post-” here refers to periods of time after a particular changed insulin dosage recommendation is generated. Similarly, the post-change data 402 can include data from the most recent two weeks since the current recommendation was generated (e.g., data from 0-2 weeks back in time). Each of the pre-change data 400 and the post-change data 402 can include one or more types of information. Here, the pre-change data 400 includes a graph 404 and percentiles 406. The graph 404 can show a blood glucose level over a period of time associated with the pre-change data 400. The percentiles 406 can show the percentage of time that the person with diabetes spent, during the relevant time period, in various ranges of blood sugar levels. For example, the percentiles 406 can reflect what percent of time was spent below a lower blood sugar threshold (e.g., 70 mg/dl), what percent of time was spent within an intermediate range of blood sugar threshold (e.g., 70-180 mg/dl), and what percent of time was spent above an upper blood sugar threshold (e.g., 180 mg/dl). Here, the post-change data 402 includes a graph 408 and percentiles 410. The graph 408 can show a blood glucose level over a period of time associated with the post-change data 402. The percentiles 410 can show the percentage of time that the person with diabetes spent, during the relevant time period, in various ranges of blood sugar levels. For example, the percentiles 410 can reflect what percent of time was spent below a lower blood sugar threshold (e.g., 70 mg/dl), what percent of time was spent within an intermediate range of blood sugar threshold (e.g., 70-180 mg/dl), and what percent of time was spent above an upper blood sugar threshold (e.g., 180 mg/dl). The pre-change data 400 and the post-change data 402 are examples of blood glucose profiles that can be evaluated to determine compliance with recommendations, and/or be used in training a classifier.

The compliance evaluation system 106 includes a compliance classifier 412 (e.g., the trained compliance classifier engine 304 in FIG. 3). The compliance classifier 412 can receive data regarding the person with diabetes and determine whether the person with diabetes has taken into account a changed insulin dosage recommendation in administering insulin. For example, the compliance classifier 412 evaluates the pre-change data 400 and the post-change data 402 to determine if the post-change data 402 reflects that the person with diabetes has taken into account the changed insulin dosage recommendation that was generated. The determination can involve classifying the post-change data 402 as either being compliant with the changed insulin dosage recommendation (e.g., reflecting that the person at least took the changed insulin dosage recommendation into account in administering insulin), or as not being compliant with the changed insulin dosage recommendation (e.g., reflecting that the person did not take the changed insulin dosage recommendation into account in administering insulin). The compliance classifier 412 can be trained on one or more kinds of data, including simulated data or actual patient data.

Training the compliance classifier 412 can include at least the following operations. A first set of blood glucose profiles can be generated, the first set of blood glucose profiles corresponding to first persons with diabetes who do not take into account a changed insulin dosage recommendation in administering insulin. For example, part of the blood glucose data 318 (FIG. 3) can be generated (e.g., from patient data and/or from simulated data), and can reflect the blood glucose characteristics associated with persons who do not take into account changed insulin dosage recommendations. A second set of blood glucose profiles can be generated, the second set of blood glucose profiles corresponding to second persons with diabetes who take into account the changed insulin dosage recommendation in administering insulin. For example, part of the blood glucose data 318 (FIG. 3) can be generated (e.g., from patient data and/or from simulated data), and can reflect the blood glucose characteristics associated with persons who take into account changed insulin dosage recommendations. A classifier (e.g., the compliance classifier 412) can be trained based on the first set of blood glucose profiles and the second set of blood glucose profiles. For example, the compliance classifier 412 can be trained to distinguish the blood glucose characteristics associated with persons who do not take into account changed insulin dosage recommendations from the blood glucose characteristics associated with persons who take into account changed insulin dosage recommendations.

The compliance evaluation system 106 includes an equality tester 414. In some implementations, the equality tester 414 can test the equality of continuous data distributions such as the pre-change data 400 and the post-change data 402. For example, the equality tester 414 can perform a Kolmogorov-Smirnov test (KS-test) on at least part of the pre-change data 400 and the post-change data 402. An outcome of the equality tester 414 can be used as a determination of whether a person with diabetes has taken into account a changed insulin dosage recommendation in administering insulin. An outcome of the equality tester 414 can be used for one or more other purposes, including, but not limited to, as a feature used in training the compliance classifier 412.

The compliance evaluation system 106 includes a hypothesis tester 416. In some implementations, the hypothesis tester 416 can test a hypothesis such as whether the post-change data 402 reflects that the changed insulin dosage recommendation was taken into account by the person with diabetes. For example, blood glucose values can follow a log-normal distribution, and the equality tester 414 can fit parameters of a log-normal distribution to the blood glucose values of at least part of the pre-change data 400 and the post-change data 402. An outcome of the hypothesis tester 416 can be used as a determination of whether a person with diabetes has taken into account a changed insulin dosage recommendation in administering insulin. An outcome of the hypothesis tester 416 can be used for one or more other purposes, including, but not limited to, as a feature used in training the compliance classifier 412.

The hypothesis tester 416 exemplifies use of a method that includes generating a first set of blood glucose profiles corresponding to first persons with diabetes who do not take into account a changed insulin dosage recommendation in administering insulin. For example, part of the blood glucose data 318 (FIG. 3) can be generated (e.g., from patient data and/or from simulated data), and can reflect the blood glucose characteristics associated with persons who do not take into account changed insulin dosage recommendations. The method includes generating a second set of blood glucose profiles corresponding to second persons with diabetes who take into account the changed insulin dosage recommendation in administering insulin. For example, part of the blood glucose data 318 (FIG. 3) can be generated (e.g., from patient data and/or from simulated data), and can reflect the blood glucose characteristics associated with persons who take into account changed insulin dosage recommendations. A hypothesis test can be applied (e.g., in the hypothesis tester 416) to the first set of blood glucose profiles and the second set of blood glucose profiles. For example, the hypothesis test can seek to determine whether a blood glucose profile being tested is associated with not taking into account changed insulin dosage recommendations, or whether the blood glucose profile being tested is associated with taking into account changed insulin dosage recommendations.

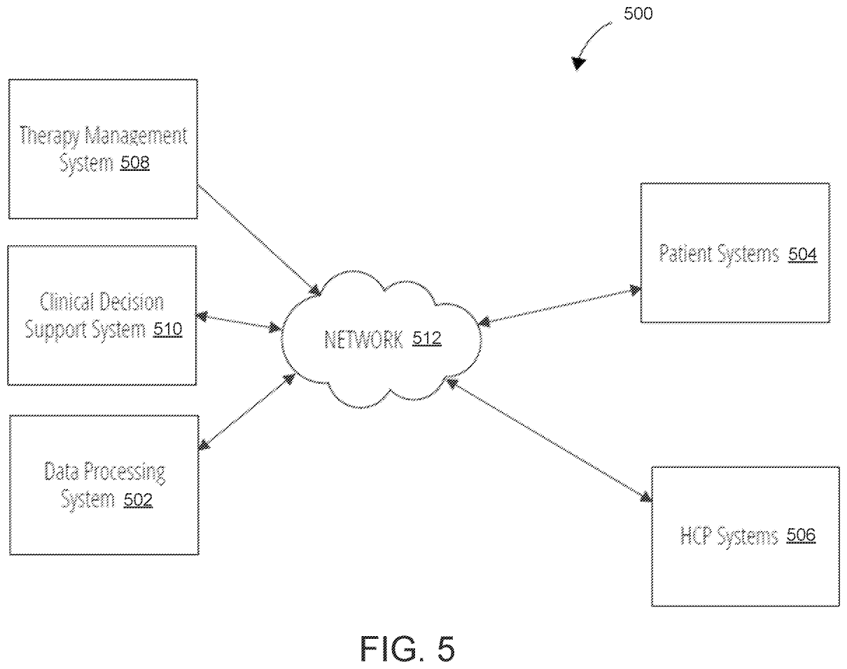

FIG. 5 shows a simplified block diagram of an example of a system 500 for insulin-based management of diabetes. Any suitable technique used by one of ordinary skill in the art in the field of data science to calculate and/or express probabilities may be used with disclosed embodiments. Some embodiments relate, generally, to insulin therapy systems and elements thereof that incorporate systems, methods and devices for TDBD-mismatch determination. The system 500 can be used with one or more other examples described elsewhere herein.

In the embodiment shown in FIG. 5, data processing system 502, clinical decision support system 510, and therapy management system 508 are computing platforms configured, generally, to provide various services related to insulin therapy, in whole or in part, to each other and to HCP systems 506 and patient systems 504. The data processing system 502, clinical decision support system 510, therapy management system 508, HCP systems 506, and patient systems 504 can be connected by one or more networks 512 (e.g., the Internet). HCP systems 506 may include, for example, portals, dashboards, electronic medical record systems, computing platforms executing the same, and more.

In some embodiments, therapy management system 508 may be one or more computing platforms configured to receive and store therapy data (such as therapy data 312 in FIG. 3) and physiological parameters about patients, issues alarms and alerts, and manages therapy settings for insulin delivery systems—all related to insulin-based management of a PWD's diabetes.

In some embodiments, clinical decision support system 510 may be one or more computing platform configured as a health data technology system for assisting HCPs with clinical decision making tasks, and more specifically in this example, assist HCPs with clinical decision making tasks related to a PWDs insulin therapy. In disclosed embodiments, clinical decision support system 510 is configured to assist with insulin-based management of diabetes, and automatically analyzes therapy data 312 (FIG. 3), identifies clinically relevant patterns in a PWD's therapy from therapy data 312, and provides data and recommendations to HCP systems 506 based on those patterns. A goal of embodiments of clinical decision support system 510 is to improve outcomes for PWDs by facilitating communication of clinically relevant “insights” about a PWD's insulin-based therapy to patient systems 504 and HCP systems 506 as well as by facilitating communication of therapy related advice from HCP systems 506 to patient systems 504.

Data processing system 502 may be one or more computing platforms configured to process therapy data 312 (FIG. 3) stored at, or received from, therapy management systems 508 and/or clinical decision support system 510. In one embodiment, data processing system 502 may, among other things, include one or more elements of computing platform 300, including trained compliance classifier engine 304 and/or trained TDBD regressor 305. In this manner, data processing system 502 may be configured to perform compliance evaluation for therapy management system 508 and/or clinical decision support system 510.

By way of example, data processing system 502 may perform compliance determination on therapy data 312 (FIG. 3) stored at clinical decision support system 510 and provide one or more reports 320 (FIG. 3) detailing one or more labeled retrospective time periods, as well as one or more determinations regarding compliance with changed insulin dosage recommendations. Clinical decision support system 510 may use the data in reports 320 (FIG. 3) to trigger insights and/or recommendations that it sends to HCP systems 506. Upon HCP system 506 accessing messages from clinical decision support system 510, data from reports 320 (FIG. 3) may be included in such message or accessible by HCP systems 506, such as if HCP systems 506 requests data to support an insight or recommendation described in a message.

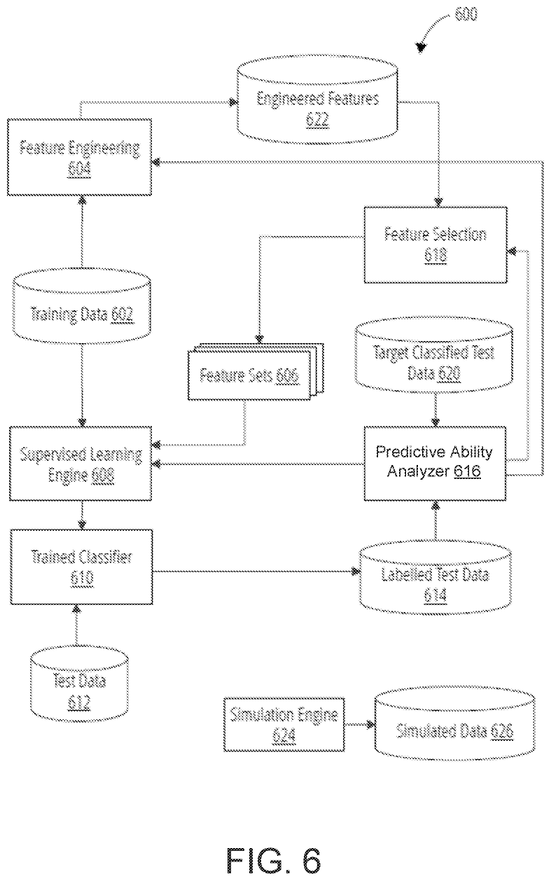

FIG. 6 shows a functional block diagram of system 600 for training a classifier (such as trained compliance classifier engine 304 in FIG. 3) and/or a regressor (such as trained TDBD regressor 305 in FIG. 3) using machine learning techniques, in accordance with disclosed embodiments. The system 600 can be used with one or more other examples described elsewhere herein.

In a contemplated operation, supervised learning engine 608 trains classifier 610 using training data 602 and sets of engineered features (i.e., feature sets 606) selected for model training purposes. In some embodiments, trained classifier 610 is a function or algorithm that determines a TDBD mismatch. An initial “best guess” may be used for trained classifier 610 which is then continually improved by supervised learning engine 608. In disclosed embodiments, trained classifier 610 and supervised learning engine 608 may implement any suitable supervised learning algorithms and ensemble methods thereof for performing embodiments of the disclosure, including, for example, decision trees, random forests, neural networks, or logistic regression. In some implementations, automated feature selection can be used to select the best model. For example, forward or backward elimination, principal component analysis, correlation-based feature selection (CFS) subset evaluation, information gain, etc. Disclosed embodiments may also implement supervised learning algorithm(s) that do not use feature selection, including, for example, one class support vector machine (SVM) without feature selection.

In one embodiment, training data 602 is labeled therapy data associated with one or more PWDs. PWDs may be chosen so they are representative of a desired domain of PWD physiologies, eating behaviors, exercise behaviors, sleeping behaviors, diurnal profile variation, and more.

In disclosed embodiments, feature sets 606 are sub-sets of features engineered (i.e., formed) in the training data 602 and used by supervised learning engine 608 to train any classifier. In one embodiment, feature sets 606 are created using a feature selection process for selecting a subset of features included in a feature domain created using feature engineering techniques. Features in a feature domain may include, for example, one or more of the features identified in the table below:

TABLE I

| Feature | Description |
|---|---|
| tl54 | time below a blood glucose value of 54 |
| tl70 | time below a blood glucose value of 70 |
| tir | time within a blood glucose range |
| tg180 | time above a blood glucose value of 180 |
| tg250 | time above a blood glucose value of 250 |
| a1c | patient identifier |
| geoMean | a geometric mean of blood glucose values |
| geoStd | A geometric standard deviation of the blood |
| | glucose values |
| loss | a loss function regarding the blood glucose |
| | values |
| tdbd_programmed | a programmed TDBD value |
| cr_programmed | a programmed carbohydrate-to-insulin ratio |
| isf-programmed | a programmed insulin sensitivity factor |
| muStarOvernight | a geometric mean of blood glucose values |
| | overnight |
| sigmaStarOvernight | A geometric standard deviation of the blood |
| | glucose values overnight |
| muStarAfterMeal | a geometric mean of blood glucose values |
| | after a meal |
| sigmaStarAfterMeal | a geometric standard deviation of the blood |
| | glucose values after a meal |
| muStarAfterCorrection | a geometric standard deviation of the blood |
| | glucose values after a correction |
| KS-test | A KS-test comparing glucose distribution |
| | before and after the settings change cause the |
| | p-value to change |
| insulin_dose | an ingested insulin dose amount |
| change in [X] | a change in any engineered feature prior to |
| | and after a new/adjusted recommendation, |
| | where [X] represents any feature in the |
| | Feature column of this Table I |

That is, one or more feature sets can be constructed, such as by a selection process at a feature engineering stage. The feature selection process can be performed using the feature sets and the data to obtain a training feature set. Feature sets 606 may be selected from the feature domain using any suitable feature selection technique or combination of techniques for trying features in the feature domain and identifying important features, including, for example, sequential forward feature selection, sequential backward elimination, and tree-based feature selection algorithms.

Labeled test data 614 is test data 612 classified and labeled by a trained classifier 610 during successive iterations of system 600. The system 600 includes target classified test data 620. Labelled test data 614 is the “true” or “target” labels for test data 612. Stated another way, it is the labeling result that is the target for trained classifier 610. Predictive ability analyzer 616 assess the predictive ability of trained classifier 610 by comparing the labels of the regressed test data 614 that were predicted by the trained classifier 610 to the true values in the target test data. Any suitable technique for calculating and/or assessing validity of a model may be used by predictive ability analyzer 616, including for example, precision, recall, number of detected events versus number of true events, confusion matrix, area-under-the-free-curve (AUC), receiver operating characteristics (ROC) curve. Techniques such as Grid search combined with cross validation, and N-fold cross-validation can be used for hyper parameter tuning of a regressor.

Feature selection 618 receives assessment results from predictive ability analyzer 616 and, in response, changes feature sets 606 to attempt to improve accuracy and/or attempt to simplify feature sets 606. As non-limiting examples, changes to feature sets 606 may include, changing weighting for features of feature sets 606, adding features to feature sets 606 to attempt to improve accuracy of predictions, removing unnecessary features from feature sets 606, and combinations thereof.

Feature engineering 604 receives assessment results from predictive ability analyzer 616 and, in some cases, performs feature engineering techniques to extract new features from test data 612 and add those features to engineered features 622. These new features may be used in the feature selection process by feature selection 618.

In one embodiment, system 600 may include simulation engine 624 configured to generate simulation data 626 from which training data 602 and test data 612 may be obtained. Simulation engine 624 may be configured to simulate insulin therapy scenarios for a variety of PWDs. PWD profiles are created that represent a cross-section of PWDs in terms of characteristics such as physiology (e.g., age, weight, height, complicating health conditions, diurnal profile variation, etc.), lifestyle (e.g., eating behaviors, exercise behaviors, diurnal variations, sleeping behaviors, meal sizes, meal times, etc.), socio-economic factors (e.g., income, race, geographic location, marriage status, child status, etc.), differences in how PWDs measure and track meal intake, differences in the operation and quality of insulin delivery systems and components the PWDs use, physiology parameters (e.g., TDBD, CR, correction factor), mismatches in physiology parameters used for dosing compared to the true underlying parameters, carbohydrate counting errors, blousing behavior (e.g., missed meals or missed boluses), CGM errors, insulin delivery pathway faults, and/or unmeasured physiological factors affecting blood glucose (as a lump model) (e.g., illness, other medications, etc.). In one embodiment, simulation engine 624 is configured to model for missing therapy data due to, for example, lost components, failure to input therapy related data, failure to wear a glucose monitor, and lost Bluetooth connection.

One or more actions can be taken in response to determining whether the person with diabetes has taken into account the first changed insulin dosage recommendation in administering insulin. When the determination indicates that the person with diabetes has taken into account the first changed insulin dosage recommendation, the action can include generating another changed insulin dosage recommendation to the person with diabetes. For example, the diabetes therapy recommendation system 104 (FIG. 1) can continue to generate insulin dosage recommendations to the person, including, if applicable, a changed insulin dosage recommendation. When the determination indicates that the person with diabetes has not taken into account the first changed insulin dosage recommendation, the action can include preventing another changed insulin dosage recommendation from being generated to the person with diabetes. For example, the diabetes therapy recommendation system 104 (FIG. 1) can cease to generate insulin dosage recommendations to the person, including, any changed insulin dosage recommendations. When the determination indicates that the person with diabetes has not taken into account the first changed insulin dosage recommendation, the action can include generating a communication without another changed insulin dosage recommendation. For example, the diabetes therapy recommendation system 104 (FIG. 1) can generate a so-called insight that addresses the current situation (e.g., by stating that it appears the person with diabetes needs to ingest higher or lower doses of insulin), but without generating a numerical insulin dosage recommendation. Such an insight can, appropriately worded, be generated to the person with diabetes or to the person's physician.

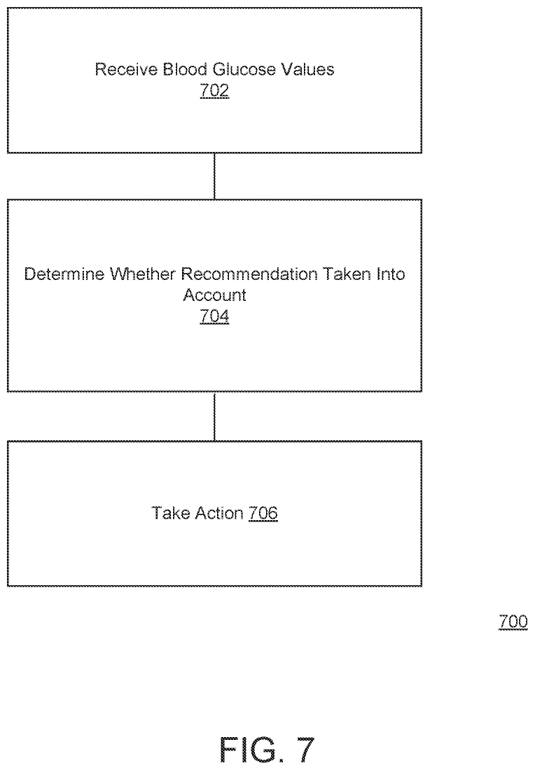

FIG. 7 shows an example of a method 700. The method 700 can be used with one or more other examples described elsewhere herein. The method 700 can include more or fewer operations than shown. Two or more operations can be performed in a different order unless otherwise indicated.

At 702, blood glucose values for a person with diabetes can be received. In some implementations, no detection is made of the amounts of insulin ingestion by the person with diabetes. The blood glucose values can be detected using a CGM or a BGM, to name just two examples. The blood glucose values relate to a time period. Particularly, during the time period a changed insulin dosage recommendation was made to the person with diabetes. As such, some of the blood glucose values can relate to times before the changed insulin dosage recommendation, and others of the blood glucose values can relate to times after the changed insulin dosage recommendation.

At 704, it can be determined whether the person with diabetes has taken into account the changed insulin dosage recommendation in administering insulin. In some implementations, the notion of taking the changed insulin dosage recommendation into account includes, but is not limited to, whether the person with diabetes is following the changed insulin dosage recommendation. For example, the person with diabetes may take the changed insulin dosage recommendation into account in making a change in insulin dosage without necessarily following the changed insulin dosage recommendation. A trained classifier can evaluate the blood glucose data to make such a determination. For example, one or more of the features listed in Table I above can be taken into account.

At 706, an action can be taken based on the determination. In some implementations, the action can include changing an insulin dosage recommendation. In some implementations, the action can include preventing another changed insulin dosage recommendation from being generated to the person with diabetes. In some implementations, the action can include generating a communication without another insulin dosage recommendation.

A trained regressor can be used in the performance of the method 700. In some implementations, the trained TDBD regressor 305 (FIG. 3) can be used in determining whether a person with diabetes has taken into account a changed insulin dosage recommendation in administering insulin. The trained TDBD regressor 305 can predict, using the blood glucose values, at least a first TDBD mismatch value corresponding to time before the change and at least a second TDBD mismatch value corresponding to time after the change. In some implementations, if the first and second TDBD mismatch values are sufficiently different from each other, then the determination is made that the person with diabetes has taken into account a changed insulin dosage recommendation in administering insulin. For example, a hypothesis test can be performed to determine if the first and second TDBD mismatch values are sufficiently different from each other.

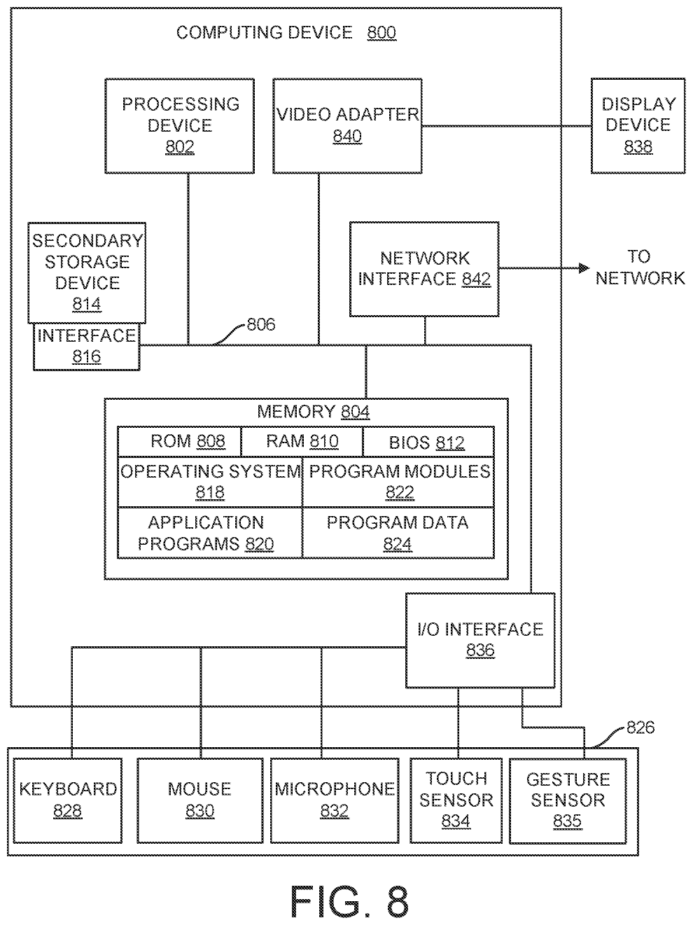

FIG. 8 illustrates an example architecture of a computing device 800 that can be used to implement aspects of the present disclosure, including any of the systems, apparatuses, and/or techniques described herein, or any other systems, apparatuses, and/or techniques that may be utilized in the various possible embodiments.

The computing device illustrated in FIG. 8 can be used to execute the operating system, application programs, and/or software modules (including the software engines) described herein.

The computing device 800 includes, in some embodiments, at least one processing device 802 (e.g., a processor), such as a central processing unit (CPU). A variety of processing devices are available from a variety of manufacturers, for example, Intel or Advanced Micro Devices. In this example, the computing device 800 also includes a system memory 804, and a system bus 806 that couples various system components including the system memory 804 to the processing device 802. The system bus 806 is one of any number of types of bus structures that can be used, including, but not limited to, a memory bus, or memory controller; a peripheral bus; and a local bus using any of a variety of bus architectures.

Examples of computing devices that can be implemented using the computing device 800 include a desktop computer, a laptop computer, a tablet computer, a mobile computing device (such as a smart phone, a touchpad mobile digital device, or other mobile devices), or other devices configured to process digital instructions.

The system memory 804 includes read only memory 808 and random access memory 810. A basic input/output system 812 containing the basic routines that act to transfer information within computing device 800, such as during start up, can be stored in the read only memory 808.

The computing device 800 also includes a secondary storage device 814 in some embodiments, such as a hard disk drive, for storing digital data. The secondary storage device 814 is connected to the system bus 806 by a secondary storage interface 816. The secondary storage device 814 and its associated computer readable media provide nonvolatile and non-transitory storage of computer readable instructions (including application programs and program modules), data structures, and other data for the computing device 800.

Although the example environment described herein employs a hard disk drive as a secondary storage device, other types of computer readable storage media are used in other embodiments. Examples of these other types of computer readable storage media include magnetic cassettes, flash memory cards, digital video disks, Bernoulli cartridges, compact disc read only memories, digital versatile disk read only memories, random access memories, or read only memories. Some embodiments include non-transitory media. For example, a computer program product can be tangibly embodied in a non-transitory storage medium. Additionally, such computer readable storage media can include local storage or cloud-based storage.

A number of program modules can be stored in secondary storage device 814 and/or system memory 804, including an operating system 818, one or more application programs 820, other program modules 822 (such as the software engines described herein), and program data 824. The computing device 800 can utilize any suitable operating system, such as Microsoft Windows™, Google Chrome™ OS, Apple OS, Unix, or Linux and variants and any other operating system suitable for a computing device. Other examples can include Microsoft, Google, or Apple operating systems, or any other suitable operating system used in tablet computing devices.

In some embodiments, a user provides inputs to the computing device 800 through one or more input devices 826. Examples of input devices 826 include a keyboard 828, mouse 830, microphone 832 (e.g., for voice and/or other audio input), touch sensor 834 (such as a touchpad or touch sensitive display), and gesture sensor 835 (e.g., for gestural input. In some implementations, the input device(s) 826 provide detection based on presence, proximity, and/or motion. In some implementations, a user may walk into their home, and this may trigger an input into a processing device. For example, the input device(s) 826 may then facilitate an automated experience for the user. Other embodiments include other input devices 826. The input devices can be connected to the processing device 802 through an input/output interface 836 that is coupled to the system bus 806. These input devices 826 can be connected by any number of input/output interfaces, such as a parallel port, serial port, game port, or a universal serial bus. Wireless communication between input devices 826 and the input/output interface 836 is possible as well, and includes infrared, BLUETOOTH® wireless technology, 802.11a/b/g/n, cellular, ultra-wideband (UWB), ZigBee, or other radio frequency communication systems in some possible embodiments, to name just a few examples.

In this example embodiment, a display device 838, such as a monitor, liquid crystal display device, projector, or touch sensitive display device, is also connected to the system bus 806 via an interface, such as a video adapter 840. In addition to the display device 838, the computing device 800 can include various other peripheral devices (not shown), such as speakers or a printer.

The computing device 800 can be connected to one or more networks through a network interface 842. The network interface 842 can provide for wired and/or wireless communication. In some implementations, the network interface 842 can include one or more antennas for transmitting and/or receiving wireless signals. When used in a local area networking environment or a wide area networking environment (such as the Internet), the network interface 842 can include an Ethernet interface. Other possible embodiments use other communication devices. For example, some embodiments of the computing device 800 include a modem for communicating across the network.

The computing device 800 can include at least some form of computer readable media. Computer readable media includes any available media that can be accessed by the computing device 800. By way of example, computer readable media include computer readable storage media and computer readable communication media.

Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 800.

Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 8 is also an example of programmable electronics, which may include one or more such computing devices, and when multiple computing devices are included, such computing devices can be coupled together with a suitable data communication network so as to collectively perform the various functions, methods, or operations disclosed herein.

The terms “substantially” and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. Also, when used herein, an indefinite article such as “a” or “an” means “at least one.”

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

A number of implementations have been described. Nevertheless, it will be understood that various modifications may be made without departing from the spirit and scope of the specification.

In addition, the logic flows depicted in the figures do not require the particular order shown, or sequential order, to achieve desirable results. In addition, other processes may be provided, or processes may be eliminated, from the described flows, and other components may be added to, or removed from, the described systems. Accordingly, other implementations are within the scope of the following claims.

While certain features of the described implementations have been illustrated as described herein, many modifications, substitutions, changes and equivalents will now occur to those skilled in the art. It is, therefore, to be understood that appended claims are intended to cover all such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components and/or features of the different implementations described.

What is claimed is:

1. A method comprising:
receiving, using a computer system and for a person with diabetes for whom amounts of insulin ingestion is not detected, blood glucose values for a time period, wherein a first changed insulin dosage recommendation was issued to the person with diabetes during the time period;
determining, using the computer system and based on the blood glucose values, whether the person with diabetes has taken into account the first changed insulin dosage recommendation in administering insulin; and
taking an action based on the determination.

2. The method of claim 1, wherein the determination indicates that the person with diabetes has taken into account the first changed insulin dosage recommendation, and wherein the action comprises generating a second changed insulin dosage recommendation to the person with diabetes.

3. The method of claim 1, wherein the determination indicates that the person with diabetes has not taken into account the first changed insulin dosage recommendation, and wherein the action comprises preventing a second changed insulin dosage recommendation from being generated to the person with diabetes.

4. The method of claim 1, wherein the determination indicates that the person with diabetes has not taken into account the first changed insulin dosage recommendation, and wherein the action comprises generating a communication without a second changed insulin dosage recommendation.

5. The method of claim 4, wherein the communication is generated to the person with diabetes or to a physician.

6. The method of claim 1, wherein the blood glucose values include a first blood glucose profile from before the first changed insulin dosage recommendation was generated, and a second blood glucose profile from after the first changed insulin dosage recommendation was generated.

7. The method of claim 6, further comprising determining, based on the first blood glucose profile and the second blood glucose profile, percentiles of the blood glucose values for the time period.

8. The method of claim 6, wherein determining whether the person with diabetes has taken into account the first changed insulin dosage recommendation in administering insulin comprises applying an equality test to the first blood glucose profile and the second blood glucose profile.

9. The method of claim 1, wherein determining whether the person with diabetes has taken into account the first changed insulin dosage recommendation in administering insulin comprises using a trained classifier.

10. The method of claim 1, wherein determining whether the person with diabetes has taken into account the first changed insulin dosage recommendation in administering insulin comprises applying a hypothesis test to the blood glucose values.

11. The method of claim 10, wherein applying the hypothesis test comprises fitting parameters of a log-normal distribution to the blood glucose values.

12. The method of claim 1, wherein determining whether the person with diabetes has taken into account the first changed insulin dosage recommendation in administering insulin comprises looking for a change in the blood glucose values opposite to a direction of the first changed insulin dosage recommendation.

13. The method of claim 1, further comprising:
predicting a first total daily basal dose (TDBD) mismatch value using at least some of the blood glucose values, the first TDBD mismatch value corresponding to time before the first changed insulin dosage recommendation;
predicting a second TDBD mismatch value using at least some of the blood glucose values, the second TDBD mismatch value corresponding to time after the first changed insulin dosage recommendation; and
determining whether the first and second TDBD mismatch values are different from each other, wherein the determination whether the person with diabetes has taken into account the first changed insulin dosage recommendation in administering insulin is based on the determination whether the first and second TDBD mismatch values are different from each other.

14. The method of claim 13, further comprising performing a hypothesis test to determine whether the first and second TDBD mismatch values are different from each other.

15. A method comprising:
generating, using a computer system, a first set of blood glucose profiles corresponding to first persons with diabetes who do not take into account a changed insulin dosage recommendation in administering insulin;
generating a second set of blood glucose profiles corresponding to second persons with diabetes who take into account the changed insulin dosage recommendation in administering insulin; and
training a classifier using the computer system, the classifier trained based on the first set of blood glucose profiles and the second set of blood glucose profiles.

16. The method of claim 15, further comprising obtaining the first set of blood glucose profiles and the second set of blood glucose profiles by simulation.

17. The method of claim 15, further comprising performing feature engineering regarding the first set of blood glucose profiles and the second set of blood glucose profiles, the feature engineering taken into account in training the classifier.

18. The method of claim 15, further comprising applying an equality test to the first set of blood glucose profiles and the second set of blood glucose profiles, and designating an outcome of the equality test as a feature in training the classifier.

19. A method comprising:
generating, using a computer system, a first set of blood glucose profiles corresponding to first persons with diabetes who do not take into account a changed insulin dosage recommendation in administering insulin;
generating a second set of blood glucose profiles corresponding to second persons with diabetes who take into account the changed insulin dosage recommendation in administering insulin; and
applying, using the computer system, a hypothesis test to the first set of blood glucose profiles and the second set of blood glucose profiles.

20. The method of claim 19, wherein applying the hypothesis test comprises fitting parameters of a log-normal distribution to the first set of blood glucose profiles and the second set of blood glucose profiles.

21. The method of claim 19, further comprising training a classifier based on an outcome of applying the hypothesis test.

22. The method of claim 19, further comprising obtaining the first set of blood glucose profiles and the second set of blood glucose profiles by simulation.
